(19) United States
(12) Patent Application Publication
(10) Pub. No.: US 2002/0133213 A1
(43) Pub. Date: Sep. 19, 2002

(54) Automotive therapeutic heat pad

(76) Inventor: Joseph A. Tippitt, Maryville, TN (US)

Correspondence Address: Joseph A. Tippitt, 3817 Fairfield Dr., Maryville, TN, 37804, US

(21) Appl. No.: 10/097,961

(22) Filed: Mar. 14, 2002

Related U.S. Application Data
(60) Provisional application No. 60/276,166, filed on Mar. 15, 2001.

(51) Int. Cl. A61F 7/00; A61F 7/12

(52) U.S. Cl. 607/98; 607/96; 607/108

(57) ABSTRACT

A portable therapeutic heat device having an electrical heating pad suitable for positioning against a users back and having electical connector means for connecting the pad to any desired electrical power source, wherein the device has pad support members affixed to the pad, preferably removable for vertical adjustment, and having upper hook-like segments adapted to hook over the rear of a seat or over a users shoulders such that the pad will be retained in a desired position or posture with respect to the users back.

This application claims priority under 35 U.S.C. 119(e)(1) based on Applicant's Provisional U.S. Patent Application Ser. No. 60/276,166, filed Mar. 15, 2001 and titled “Automotive Heat Pad”.

### BACKGROUND OF THE INVENTION

1. Field

This invention concerns a heating pad device designed to provide therapeutic heat to at least major portions of a users back while the user is sitting on an automobile seat or in a chair, sofa, couch or the like, or even while standing, The device provides many features such as temperature regulation, both auto battery and home electrical power accommodation, position adjustability of the pad up and down relative to the persons back, prevention of inopportune displacement of the device when affixed to the seat or to a persons body, and unique structure for quickly and easily adjusting the position of the device On a scat and for installing the device on a seat or persons body or removing it therefrom.

Driving an automobile or being a passenger places a great deal of strain on the lower back than does standing or lying down, Sitting in the same position for extended periods of time can strain the spine and impair blood circulation. In many cases the muscles will spasm as a result of the reduced flow of oxygen-rich blood. The present automotive heating pad device gets warm enough to increase circulation and help relax spasmed muscles, particularly while driving.

2. Prior Art

Heretofore and exemplified by the following U.S. patents many and various types of automotive therapeutic heating devices have been proposed: U.S. Pat. Nos. 3,027,493; 4,629,868; 4,628,188; 4,335,725; 4,633,061; 4,149,066; 3,852,570; 6,073,998; 5,998,770; 5,288,974; 4,245,149; 4,825,048; 4,952,776; and 4,695,091, the incorporation herein of their disclosures by reference in their entireties is hereby made. Such devices typically are used for warning automotive seats for per se, or for use by occupants of the vehicle by placement of heating pads on the vehicle seat or behind the back of the occupant.

Some of these prior pads are quite flexible such that they are easily moved from one position to another on the vehicle seat and/or against the users body to accommodate the special comfort needs of particular occupants. Such repositioning of the pad as well as natural sliding or folding or the like movement of the pad, particularly when the user gets off of the seat and out of the vehicle, or where the user simply wants to reposition himself on the seat, causes the pad to crumple, crinkle, fold up or the like which presents difficulties to the user in replacing the pad behind the users back in a desired position, particularly vertically.

Others of these prior pads such as shown in U.S. Pat. No. 4,335,725 are designed primarily for heating and/or supporting certain regions of a persons back such as the lumbar region. The bulkiness and configuration of such a device however, does not prevent its from a desired position as described above and further restricts its positioning on the vehicle seat and essentially prevents its use on upper regions of the back.

It is evident from the aforesaid prior art that a need for and thus objectives exists for the development of a therapeutic heat pad device for use against a users back, which device does not significantly change its shape or its desired posture with respect to the users back when moved by the user along a vehicle seat or when it is moved from one seat in the vehicle to another, or even when it is removed from the vehicle and thereafter repositioned on the vehicle great or on a chair, sofa, couch or the like in a home. Also, it is desirable that the pad height relative to the seat back can readily be adjusted to impact on any desired portion of the users back without having to heat the entire back, and further that the device be provided with structure for retaining itself in a desired position on a seat or on a persons body such that, for example, movement of the user such as leaning forward or to the side or even standing up will not disarrange or dislodge the pads.

### SUMMARY OF THE INVENTION

The above and other objectives hereinafter appearing have been attained in accordance with the present invention thru the discovery of the structure of a portable therapeutic device have an electrical heating pad suitable for positioning against a users back and having electrical connector means for connecting the pad to any desired electrical power source, but preferably a 12 volt source such as an automobile, wherein the device has pad support means affixed to the pad, preferably removable for vertical adjustment, and having upper hook-like segments adapted to hook over the rear of a seat or over a users shoulders such that the pad will be retained in a desired position or posture with respect to the users back.

### BRIEF DESCRIPTION OF THE DRAWINGS

The invention will be understood further from the drawings herein and description, wherein the figures are not drawn to scale or proportion, and wherein:

FIG. 1 is a prospective view of the present device depicted by a portion of the auto seat heating pad of the aforementioned U.S. Pat. No. 3,017,493 with the present pad support means affixed thereto in position to be hooked over a users shoulders, wherein the basic construction of the pad of said patent is useful with the present invention;

FIG. 2 is an enlarged view of the heating element of FIG. 1;

FIG. 3 is a view of the back of the pad of FIGS. 1 and 2 showing a preferred placement thereon of a hook and loop (Velcro) component;

FIG. 4 is a view as in FIG. 3 with the pad support means attached thereto by mating Velcro components;

FIG. 5 is a partial cross-sectional view taken along line 5-5 of FIG. 4 with the present device mounted on the back of a vehicle bucket seat and with the Velcro components exaggerated in thickness for clarity;

FIG. 6 is a front view of the vehicle bucket seat of FIG. 5 with the present device mounted on the back thereof; and

FIG. 7 is a cross-sectional view of approximately twice the actual size of an off-the-shelf four wire electrical cable preferable for use as the pad supports in the present invention.

### DETAILED DESCRIPTION

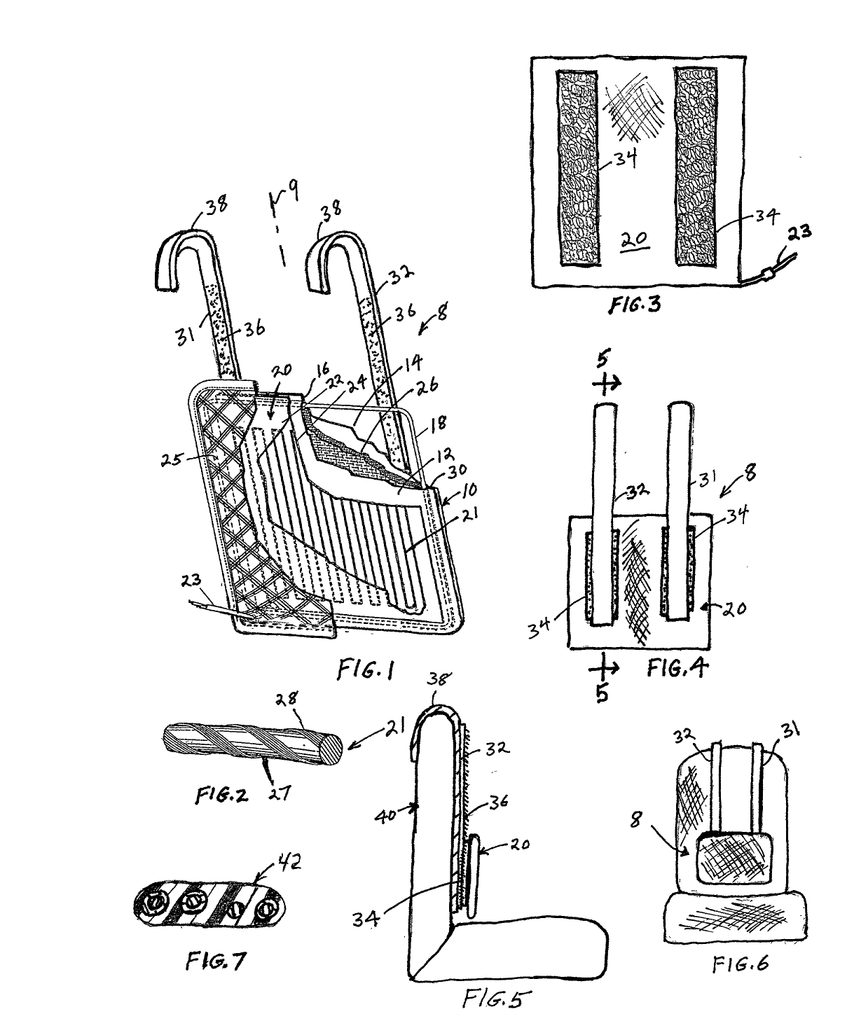

With reference to the present drawings, to U.S. Pat. No. 3,017,493 with renumbering and minor changes to the drawing in FIG. 1 thereof being shown in FIG. 1 hereof, and to the claims herein, the present portable heat pad device 8 having a vertical axis 9 comprises a base 10 comprised of heat conductive but electrically non-conductive, flexible panels 12 and 14 which are joined, e.g., adhesively or stitched together all the way around their edges as at 16. These panels may be comprised of natural or synthetic fibrous fabric material or plastic sheet material such as polyvinyl, polyolefin or polyester. A rectangular wire frame 18 may be secured within the interior perimeter of base 10 to provide any desired stiffness to the base. Secured within base 10 is a heating pad generally designated 20 formed of two sheets 22 and 24 of heat conductive but electrically non-conductive material as described above for panels 12 and 14 which are adhesively joined together as at 30. A heating element 21 is arranged in sinuous fashion between sheets 22 and 24.

The ends of the heating element 21 are connected to an electric cord 23 which is provided with a usual plug so that it can be attached to a suitable outlet such as the cigarette lighter socket located on the automobile instrument panel. The base 10 is enclosed within a heat conductive cover 25 which may have decorative qualities.

A desired thickness of electrically non-conductive but heat conductive cushion material such as 26 may be placed in base 10, e.g., adjacent to the outer surfaces of sheets 22 and 24. A preferred thickness is a total from about ¼ in. to about 1.0 in.

As stated in said U.S Pat. No. 3,017,493, and as shown in FIG. 2 the heating element 21 may consist of a thin yarn core 27 such as nylon, cotton, fiber glass or other flexible material non-conductive to electricity, and which has a smooth slippery surface. A heating element conductor 28 consisting of six or more thin strands, in side-in-side relation, are wound tangentially in helicoid fashion around the core. Many types of resistance heating pads and heating elements are available in the market place and the present invention has wide applicability thereto.

The present pad support means in one preferred embodiment comprises a pair of spaced elongated members 31, 32 of any sturdy laterally spaced and affixed at their lower portions to the heating pad by, e.g., Velcro, mating hook and loop material components 34, 36, a different one of said components being permanently affixed to said members and to said pad. The component affixed to the members preferably extends upwardly thereon as shown such that the pad can be placed in any vertical position on a users back. The pad, of course, may be permanently affixed to the members in a desired vertical location.

The upper portion of each said member is provided with a hook segment 38 for hooking over a seat back 40 as shown in FIG. 5 or, when inverted, for hooking over a persons shoulders. These members are comprised preferably of multi-wire, insulated electrical cable 42 as shown, for example, in cross-section in FIG. 7. Such cable is easily deformed by hand, i.e., twisted, bent or the like, such that the hook segment 38 can be contoured to snugly fit over the top of the seat back or over an individuals shoulders with the heat pad lying adjacent the users back. It is noted that such deformable members are sufficiently strong as to support even floppy heat pads while allowing the users weight pressed against the device to readily deform the members to comfortably fit the contour of the users back.

The invention has been described in detail with particular reference to preferred embodiments thereof, but it will be understood that variations and modifications will be effected with the spirit and scope of the invention.

I claim:

1. A portable heat pad device comprising an electrical heating pad means having a substantially vertical axis, a body contact front portion, a rear seat back contact portion, said portions being of heat conductive but electricity non-conductive cushion or upholstery material, and electrical resistance heat wiring sandwiched between said portions, manually operable control means for regulating electrical current flow to said wiring to cause said pad means to become heated to a selected temperature above body temperature and within a comfortable temperature range, support means affixed to said pad means for supporting the same in a substantially vertical orientation on the back of a seat means or on a persons back, said support means having upper hook segments adapted to hook over the rear of a seat means or over a users shoulders to maintain said pad means in a desired position respectively on said seat means or on said users back.

2. The device of claim 1 wherein said pad means has a substantially flat outer contour and a substantially uniform thickness, and wherein said hook segments are comprised of semi-rigid deformable material such that a user can bend said segments into a firmly set configuration which will conform snugly to the upper portions of the back of a vehicle or other seat means, or to a users shoulders, but which segments can be rebent to other desired configurations.

3. The device of claim 2 wherein said support means comprises a pair of elongated members laterally spaced and affixed at their lower portions to said pad means, said members each being comprised of deformable metal.

4. The device of claim 3 wherein said metal is selected from the group consisting essentially of steel, brass aluminum or copper.

5. The device of claim 4 wherein said metal is in wire form and encapsulated in a flexible sheath of plastic material.

6. The device of claim 5 where said members are removably affixed to said pad means by mating hook and loop components.

7. The device of claim 6 wherein one of said components is substantially vertically positioned along each of said members to provide for vertical position adjustment of said pad means on said members.

8. The device of claim 5 wherein said members are comprised of multi-copper wire, insulated electrical cable.
